(19) United States
(12) Patent Application Publication
(10) Pub. No.: US 2024/0101679 A1
(43) Pub. Date: Mar. 28, 2024

(54) STABLE AQUEOUS BUFFER FREE FORMULATION OF AN INTEGRIN ANTIBODY

(71) Applicant: DR. REDDY’S LABORATORIES LIMITED, Hyderabad (IN)

(72) Inventors: Murali JAYARAMAN, Kancheepuram (Dist) (IN); Swapnil Vasudeo PAKHALE, Jalgaon Dist (IN); Shrija GHOSH, Lucknow (IN); Ananya SAHA, Agartala (IN)

(21) Appl. No.: 18/265,812

(22) PCT Filed: Dec. 9, 2021
(86) PCT No.: PCT/IN2021/051156

§ 371 Date: Jun. 7, 2023

(30) Foreign Application Priority Data
Dec. 9, 2020 (IN) 202041053512

(51) Int. Cl. C07K 16/28; A61K 47/10

(52) U.S. Cl. CPC C07K 16/2842; A61K 47/10; C07K 2317/94

(57) ABSTRACT

The present invention discloses a buffer free formulation of high concentration α4β7 antibody, comprising α4β7 antibody, water, and surfactant, and stabilized at a pH of 6.0-6.5. The disclosed antibody formulations are liquid formulations and can be lyophilized. Further, the said formulations are also suitable for different mode of administration such as subcutaneous/intravenous, for therapeutic use.

### FIELD OF THE INVENTION

The present invention is related to an aqueous, buffer free formulation of an antibody molecule, stabilized at a particular pH, without any buffering agent. The disclosed formulation stabilizes the antibody from about 50 mg/ml to about 200 mg/ml which are suitable for intravenous or subcutaneous route of administration.

### BACKGROUND

Over the past two decades, recombinant DNA technology has led to the commercialization of many proteins, particularly antibody therapeutics. The effectiveness of these therapeutic antibodies is majorly dependent on the stability, route of administration and their dosage forms and concentrations. This in turn, necessitates therapeutic antibodies to be formulated appropriately to retain the stability and activity of a therapeutic antibody.

Formulations for each route of administration and dosage forms may be unique and, therefore, have specific requirements. Solid dosage forms, such as lyophilized powders, are generally more stable than liquid (aqueous) formulations. However, reconstitution of the lyophilized formulation requires a significant vial overfill, care in handling and involves high production cost relative to a liquid formulation. While liquid formulations are advantageous in these and are usually preferred for injectable protein therapeutics (in terms of convenience for the end user and ease of preparation for the manufacturer), this form may not always be feasible given the susceptibility of proteins to denaturation, aggregation and oxidation under stresses such as temperature, pH changes, agitation etc. All of these stress factors could result in the loss of biological activity of a therapeutic protein/antibody. In particular, high concentration liquid formulations are susceptible to degradation and/or aggregation. Nevertheless, high concentration formulations may be desirable for subcutaneous or intravenous route of administration, as the frequency of administration and injection volume is reduced. On the other hand, specific treatment schedule and dosing might require a low concentration formulation and prefer intravenous route of administration for more predictable delivery and complete bioavailability of the therapeutic drug.

Thus, designing a formulation that is stable at high or low concentrations of the therapeutic protein/antibody, aiding in different route of administration (intravenous or subcutaneous), pose a significant developmental challenge. Further, every protein or antibody with its unique characteristics and properties of degradation, adds to the complexity in the development of a stable formulation and may demand a specific formulation. Additionally, a formulation combination with increased concentration of a therapeutic protein in a buffer, along with excipients, may increase the viscosity of the formulation and in turn increase the injection time. Further, specific buffering agents stabilizing the protein are known to result in pain at the site of injection. Hence, it is necessary to develop an improved formulation, which addresses the above difficulties in a therapeutic protein composition.

### SUMMARY

The present invention discloses a buffer free formulation of an α4β7 antibody comprising, about 50 mg/ml to about 200 mg/ml α4β7 antibody, water and surfactant. The antibody formulated in water maintains solubility as well as stability, even at high concentrations of the antibody. In another aspect, the disclosed buffer free α4β7 antibody formulations do not require any specific buffering agent to maintain/stabilize the pH of the formulation.

In particular, the invention discloses a buffer free formulation of an α4β7 antibody, comprising an α4β7 antibody, PEG, water and surfactant. The formulation is stabilized at a pH of 6.0 to 6.5. The antibody in the said formulation is stable and soluble in water, even at high concentrations. The formulations exhibit solubility and stability at room temperature and under accelerated conditions such as at 40° C. for at least one week.

The disclosed formulations and methods of the invention stabilize the α4β7 antibody in concentrations ranging from about 50 mg/ml to about 200 mg/ml.

### DETAILED DESCRIPTION OF THE INVENTION

### Definitions

The term “around,” or “about” or “approximately” shall generally mean within 20 percent, within 10 percent, within 5, 4, 3, 2 or 1 percent of a given value or range. Numerical quantities given are approximate, meaning that the term “around,” “about” or “approximately” can be inferred if not expressly stated.

The term “antibody” refers to a glycoprotein comprising at least two heavy (H) chains and two light (L) chains inter-connected by disulfide bonds, or an antigen-binding portion thereof. The “antibody” as used herein encompasses whole antibodies or any antigen binding fragment (i.e., “antigen-binding portion”) or fusion protein thereof.

The term “buffering agent” refers to an agent which resists any change in pH of a solution near a chosen value, up on addition of acid or base.

The term “stable” formulation refers to the formulation wherein the antibody therein retains its physical stability and/or chemical stability and/or biological activity. Stability of an antibody formulation is measured in terms of aggregate content and/or monomeric content and/or charge variants content of the antibody in the composition.

Stability studies provides evidence of the quality of an antibody under the influence of various environmental factors during the course of time. ICH's “Q1A: Stability Testing of New Drug Substances and Products,” states that data from accelerated stability studies can be used to evaluate the effect of short-term excursions higher or lower than label storage conditions that may occur during the shipping of the antibodies.

Various analytical methods are available for measuring the physical and chemical degradation of the antibody in the pharmaceutical formulations. An antibody “retains its physical stability” in a pharmaceutical formulation if it shows substantially no signs of aggregation, precipitation and/or denaturation upon visual examination of color and/or clarity, or as measured by UV light scattering or by size exclusion chromatography. An antibody is said to retain its “chemical stability” in a pharmaceutical formulation when its shows no or minimal formation of aggregates and/or product variants which may include variants as a result of chemical modification of antibody of interest such as deamination, oxidation etc. Analytical methods such as ion exchange chromatography and hydrophobic ion chromatography may be used to investigate the chemical product variants.

The term ‘monomer’ as used herein describes antibodies consisting of two light chains and two heavy chains. The monomer content of an antibody composition is typically analyzed by size exclusion chromatography (SEC). As per the separation principle of SEC the large molecules or molecules with high molecular weight (HMW) elute first followed by smaller or lower weight molecules. In a typical SEC profile for an antibody composition, aggregates that may include dimers, multimers, etc., elute first, followed by monomer, and the clipped antibody variants or degradants may be eluted last. In some circumstances the aggregate peak or the degradant peaks may not elute as a baseline separated peaks but instead as a shoulder or abnormal broad peaks. In order to maintain the appropriate activity of an antibody, in particular of a therapeutic antibody, it is desirable to reduce the formation of aggregate or fragmentation of products and hence control the monomer content to a target value. Ability to inhibit the formation of aggregate and degradant content as measured at various time points during stability studies may indicate the suitability of the candidate formulation for antibody of interest. TSK-GEL G3000SWXL (7.8 mm×30 cm) column from TOSCH can be used on water HPLC to perform SEC.

The term ‘main peak’ as used herein refers to the peak that elutes in abundance (major peak) during a cation exchange chromatography. The peak that elutes earlier than the main peak, during a cation exchange chromatography, with a charge that is acidic relative to the main peak is termed acidic variant peak. The peak that elutes later than the main peak, during a cation exchange chromatography, with a charge that is relatively basic than the main peak is termed as basic variant peak. The main peak content can be determined by Ion exchange chromatography (IEC). There are two modes of IEC available viz., cation and anion exchange chromatography. Positively charged molecules bind to anion exchange resins while negatively charged molecules bind to cation exchange resins. In a typical cation exchange chromatographic profile of an antibody composition acidic variants elute first followed by the main peak and thereafter lastly the basic variants will be eluted. The acidic variants are a result of antibody modifications such as deamidation of asparagine residues. The basic variants are a result of incomplete removal of C-terminal lysine residue(s). In general, in an antibody a lysine residue is present at the C-terminal end of both heavy and light chain. An antibody molecule containing lysine at both heavy and light chain is referred to as K2 variant, the antibody molecule containing lysine residue at either one of heavy and light chain is referred to as K1 variant and antibody molecule having none is K0 molecule. Carboxypeptidase B (CP-B enzyme) enzyme acts on the C-terminal lysine residues present on K2 and K1 variants and thus converting them as K0 molecules. As per circumstances of the case, the IEC analysis can be carried out for samples digested with carboxypeptidase B (CP-B) enzyme. In a typical stability study it is expected that a stable formulation leads to reduction in formation of charge variants (acidic and basic variants), during the study, and hence minimize any reduction in main peak content.

Pharmaceutically acceptable excipients refer to the additives or carriers, which may contribute to stability of the antibody in formulation. The excipients may encompass stabilizers and tonicity modifiers. Examples of stabilizers and tonicity modifiers include, but not limited to, sugars, salts, surfactants, and derivatives and combination thereof.

The term “sugar” refers to organic compounds having the general formula Cn(H2O)n. Sugars includes monosaccharaides, disaccharides.

The term “polyol” refers to an organic compound containing multiple hydroxyl groups. Examples of polyol include, sugar alcohols and polymeric polyols, such as, and not limited to, mannitol, sorbitol, xylitol, poly ethylene glycol (PEG) etc.,

Surfactant refers to pharmaceutically acceptable excipients used to protect the protein formulations against various stress conditions, like agitation, shearing, exposure to high temperature etc. The suitable surfactants include but are not limited to polyoxyethylensorbitan fatty acid esters such as Tween 20™ or Tween 80™, polyoxyethylene-polyoxypropylene copolymer (e.g. Poloxamer, Pluronic), sodium dodecyl sulphate (SDS) and the like or combination thereof.

The term “fragments” herein refers to a part of large entity such as part of protein or antibody which consists of less than the entire amino acid sequence of the protein or the antibody which are formed due to terminal or internal deletion or splicing of a portion of the protein/antibody.

The term “charge variants” herein refers to an antibody variants which has net positive or negative charge and contains either lower or higher isoelectric point (pI) than the antibody of interest. Examples of charge variants include acidic variants and basic variants. The acidic variants of an antibody can be formed due to deamidation of glutamine and aspargine and sialylation which may impart net negative charge to the antibody and resulted in decrease in pI of the antibody. The basic variants of an antibody can be formed due to C-terminal lysine variation, oxidation, glycine amidation, succinamide formation, removal of sialic acids which may impart net positive charge to the antibody and resulted in increase in pI of the antibody.

Certain specific aspects and embodiments of the invention are more fully described by reference to the following examples. However, these examples should not be construed as limiting the scope of the invention in any manner.

### DETAILED DESCRIPTION OF EMBODIMENTS

The present invention discloses a buffer free aqueous formulation of an α4β7 antibody, comprising an α4β7 antibody, water and surfactant.

In one embodiment, the invention discloses a buffer free formulation of an α4β7 antibody, stabilized at a pH of 6.0-6.5, comprising α4β7 antibody, water and surfactant.

In an embodiment, the invention discloses an aqueous formulation of α4β7 antibody, comprising an α4β7 antibody, water and surfactant, wherein the formulation is stabilized at a pH of 6.0-6.5 and is devoid of any buffering agent.

In another embodiment, the invention discloses a method of stabilizing an α4β7 antibody in an aqueous solution, comprising;
a) expressing and purifying an α4β7 antibody,
b) subjecting the purified antibody to diafiltration with a buffer free diafiltration medium comprising water to obtain the α4β7 antibody in solution,
c) ultra-filtering the diafiltered antibody solution in water, to concentrate upto 200 mg/ml,
d) followed by formulating the antibody in water, to obtain a highly concentrated α4β7 antibody solution, wherein the antibody is stable at room temperature.

In any of the above mentioned embodiments, the α4β7 antibody formulation further comprises one or more pharmaceutically acceptable excipients, and the one or more pharmaceutically acceptable excipients are polyol, salt, amino acid or surfactant.

In another embodiment, the invention discloses a method of stabilizing an α4β7 antibody in an aqueous solution, comprising;
a) expressing and purifying an α4β7 antibody,
b) subjecting the purified antibody to diafiltration with a buffer free diafiltration medium comprising water to obtain the α4β7 antibody in solution,
c) addition of poly ethylene glycol (PEG) and sodium chloride/salt to the diafiltered antibody solution;
d) ultra-filtering the antibody solution obtained from step c) to concentrate upto 200 mg/ml,
e) followed by addition of a surfactant to the concentrated antibody solution obtained from step d),
wherein the concentrated α4β7 antibody solution obtained by the said method is stable at a pH value of 6.0-6.5, and exhibits stability at room temperature.

In the above embodiment, the antibody in the formulation is stable at room temperature for 4 weeks.

In an embodiment, the invention discloses a buffer free formulation of an α4β7 antibody comprising about 50 mg/ml to about 200 mg/ml of α4β7 antibody, water, and surfactant.

In an embodiment, the invention discloses a buffer free formulation of an aqueous α4β7 antibody, comprising;
about 60 mg/ml α4β7 antibody,
water,
surfactant.

In another embodiment, the invention discloses a buffer free aqueous formulation of α4β7 antibody, comprising;
about 160 mg/ml α4β7 antibody,
water,
surfactant.

In another embodiment, the invention discloses an aqueous formulation of α4β7 antibody, stabilized at a pH of 6.0-6.5, comprising;
about 160 mg/ml α4β7 antibody,
water,
surfactant and,
wherein, the pH of the formulation is maintained without any buffering agent.

In the above mentioned embodiment, the formulation may optionally comprises poly ethylene glycol (PEG) and/or salt.

In an embodiment, the invention discloses a buffer free formulation of an α4β7 antibody, stabilized at a pH of 6.0-6.5, comprising about 50 mg/ml to about 200 mg/ml of α4β7 antibody, water, PEG, surfactant, and optionally contains amino acid and/or salts.

In any of the above said embodiments, the concentration of α4β7 antibody is 50 mg/ml, ‘or’ 60 mg/ml, ‘or’ 70 mg/ml, ‘or’ 80 mg/ml, ‘or’ 90 mg/ml, ‘or’ 100 mg/ml, ‘or’ 110 mg/ml, ‘or’ 120 mg/ml, ‘or’ 130 mg/ml, ‘or’ 140 mg/ml, ‘or’ 150 mg/ml, ‘or’ 160 mg/ml, ‘or’ 170 mg/ml, ‘or’ 180 mg/ml, ‘or’ 190 mg/ml, ‘or’ 200 mg/ml.

In any of the above embodiments, the formulation additionally contains PEG and sodium chloride.

In an embodiment, the invention discloses a buffer free formulation of an aqueous α4β7 antibody, stabilized at a pH of 6.0-6.5, comprising;
about 150 mg/ml to about 170 mg/ml of α4β7 antibody,
water,
PEG,
sodium chloride, and
surfactant

In the above said embodiment, the buffer free α4β7 antibody formulated in a composition comprising water, PEG, sodium chloride, and surfactant is soluble and exhibits stability at room temperature for at least 3 days or 7 days or 14 days or 28 days.

In an embodiment, the invention discloses an aqueous high concentration buffer free α4β7 antibody formulation, comprising about 150 mg/ml to about 170 mg/m of α4β7 antibody, PEG, arginine, salt and surfactant, at a pH of 6.0 to 6.5, and the said formulation exhibits stability at 25° C. for four weeks.

In an embodiment, the invention discloses a buffer free formulation of an α4β7 antibody, comprising about 150 mg/ml to about 170 mg/ml of α4β7 antibody, water, PEG, arginine, sodium chloride and surfactant, at a pH of 6.0 to 6.5, wherein the formulation is stable for four weeks at 40° C.

In the above said embodiment, the α4β7 antibody formulation is stable by maintaining ≥97% of the antibody in its monomeric form, when the formulation is stored at 40° C. for 4 weeks.

In an embodiment, the invention discloses a method of controlling aggregation of an α4β7 antibody in an aqueous buffer free formulation composition of the antibody, by formulating the α4β7 antibody in a composition comprising water, PEG, and surfactant, and wherein the pH of the formulation is maintained to a pH value of 6.0 to 6.5 without any buffering agent. The composition may further optionally comprise amino acid and/or salt.

In the above mentioned embodiment, concentration of antibody present in the formulation obtained by the said method is from 50 mg/ml to 200 mg/ml.

In an embodiment, the invention discloses a method of controlling aggregation in an α4β7 antibody in an aqueous buffer free formulation composition of the antibody, by formulating the antibody in a composition comprising water, PEG, arginine, salt and surfactant, and at a pH of 6.0 to 6.5, wherein the formulation is stable with the aggregate content of the antibody less than 2% when stored at 40° C. for four weeks or at 25° C. for four weeks.

In an embodiment, the invention discloses a method of reducing formation of charge variants of an α4β7 antibody in an aqueous formulation composition of the antibody, by formulating the antibody in a composition comprising water, PEG, and surfactant, and wherein the pH of the formulation is maintained to a pH value of 6.0 to 6.5, without any buffering agent and the reduction in charge variants of antibody in water based formulation is, when compared with the antibody in buffer based formulation.

In the above embodiment, the composition may optionally comprise amino acid and/or salt.

In an embodiment, the invention discloses a method of reducing formation of acidic variants of an α4β7 antibody in an aqueous formulation composition of the antibody, by formulating the antibody in a composition comprising water, and surfactant, and wherein the pH of the formulation is maintained to a pH value of 6.0 to 6.5 without any buffering agent, and the reduction in acidic variants of antibody in water based formulation is, when compared with the antibody in buffer based formulation.

In the above embodiment, concentration of α4β7 antibody present in the formulation is about 170 mg/ml.

In an embodiment, the invention discloses a method of reducing formation of acidic variants of an α4β7 antibody in an aqueous formulation composition of the antibody, by formulating the antibody in a composition comprising water, PEG, and surfactant, and wherein the pH of the formulation is maintained to a pH value of 6.0 to 6.5 without any buffering agent, and the reduction in acidic variants of antibody in water based formulation is, when compared with the antibody in buffer based formulation.

In the above embodiment, the composition may optionally comprise amino acid and/or salt.

In an embodiment, the invention discloses a method of controlling formation of acidic variants of a high concentration α4β7 antibody in an aqueous formulation composition of the antibody, by formulating the antibody in a composition comprising water, and surfactant and wherein the concentration of the antibody is about 170 mg/ml and the formulation is stable with a change in acidic variants content of the antibody is less than 1% when stored at 25° C. for one week.

In an embodiment, the invention discloses a method of controlling formation of acidic variants of an α4β7 antibody in an aqueous buffer free formulation composition of the antibody, by formulating the antibody in a composition comprising water, PEG, arginine, salt and surfactant and at a pH of 6.0 to 6.5, and wherein the formulation is stable with a change in acidic variants content of the antibody less than 10% when stored at 40° C. for four weeks or at 25° C. for four weeks.

In an embodiment, the invention discloses a method of maintaining main peak content of an α4β7 antibody in an aqueous formulation composition of the antibody, by formulating the antibody in a composition comprising water, PEG, and surfactant, and wherein the pH of the formulation is maintained to a pH value of 6.0 to 6.5 without any buffering agent.

In the above mentioned embodiment, the composition may optionally comprise amino acid and/or salt.

In the above mentioned embodiment, the buffer free formulation composition maintains 50% or more of the antibody in main peak content when the formulation is stored at 40° C. for four weeks or at 25° C. for four weeks.

In any of the above mentioned embodiments of the invention, the α4β7 antibody formulation is stable without any visible particles even under accelerated conditions.

In any of the above mentioned embodiment, the α4β7 antibody formulation exhibits colloidal stability.

In any of the above mentioned embodiments, viscosity of buffer free α4β7 antibody formulation is less as compared to the buffer based α4β7 antibody formulation.

Another aspect of the invention provides a vial, pre-filled syringe or autoinjector device, comprising any of the subject formulations described herein. In certain embodiments, the aqueous formulation stored in the vial pre-filled syringe or autoinjector device contains buffer free high concentration (˜160 mg/ml) of α4β7 antibody, water and surfactant, yet stabilized at a pH of 6.0-6.5, without any buffering agent.

In any of the above mentioned embodiments, the formulation of α4β7 antibody is a stable liquid (aqueous) formulation, which can be used for parenteral administration. Parenteral administration includes intravenous, subcutaneous, intra peritoneal, intramuscular administration or any other route of delivery generally considered to be falling under the scope of parenteral administration and as is well known to a skilled person.

In any of the above embodiments of the invention, the stable liquid/aqueous α4β7 formulation which is suitable and can be lyophilized as lyophilized powders. Further, the lyophilized formulation of α4β7 antibody can be reconstituted with appropriate diluent to achieve the liquid formulation suitable for administration.

In any of the above mentioned embodiments, the α4β7 antibody is vedolizumab.

In any of the above mentioned embodiments, stability of the antibody formulation is measured in terms of it's aggregate content or monomeric content or charge variants content.

Certain specific aspects and embodiments of the invention are more fully described by reference to the following examples. However, these examples should not be construed as limiting the scope of the invention in any manner

### EXAMPLES

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

An α4β7 antibody, vedolizumab, suitable for storage in the present pharmaceutical composition is produced by standard methods known in the art. For example, vedolizumab is prepared by recombinant expression of immunoglobulin light and heavy chain genes in a mammalian host cell such as Chinese Hamster Ovary cells. Further, the expressed vedolizumab is harvested and the crude harvest is subjected to standard downstream process steps that include purification, filtration and optionally dilution or concentration steps. For example, the crude harvest of vedolizumab may be purified using standard chromatography techniques such as affinity chromatography, ion-exchange chromatography and combinations thereof. The purified vedolizumab solution can additionally be subjected to one or more filtration steps, and the solution obtained is subjected to further formulation studies.

### Example 1: Buffer Free High Concentration Vedolizumab Formulations with PEG and Salt

To prepare a buffer free ‘high concentration vedolizumab formulation, approximately 60-70 mg/ml vedolizumab in a buffer composition comprising histidine-phosphate buffer, trehalose, arginine and surfactant obtained from downstream chromatographic steps. The obtained vedolizumab sample was buffer exchanged at least three times with a composition comprising water and 50 mM sodium chloride. Post which, 10% PEG was added to the samples followed by ultrafiltration and concentrated up to 170 mg/ml. Polysorbate-80 was added to the obtained high concentration vedolizumab formulation. The pH of the vedolizumab formulation, without any buffering agent, was found to be 6.1. Further, these high concentration vedolizumab formulation was subjected for accelerated stability conditions such as at 40° C. for one week. Post which, the samples were measured to check various quality attributes such as monomer content, low molecular weight species, acidic variant content of the antibody. Results are given in Table 1. The buffer free vedolizumab formulation is clear without any visible particles even after storage at 40° C., which itself indicates the formulation is stable.

TABLE 1
Quality attributes of buffer free vedolizumab formulation
when stored at 40° C. for one week.

| | | | Low | | | | | |
|---|---|---|---|---|---|---|---|---|
| | | | molecular | | | | | |
| | Monomer | | weight | | Acidic | | | |
| | content | | species | | variants | | pH at | |
| | at 40° C. | | at 40° C. | | at 40° C. | | 40° C. | |
| Formulation | T0 | T1 W | T0 | T1 W | T0 | T1 W | T0 | T1 W |
| 170 mg/ml | 99.0 | 96.12 | 0.05 | 0.3 | 17.13 | 17.97 | 6.1 | 6.1 |
| vedolzumab, 10% | | | | | | | | |
| PEG, 2.92 mg/ml | | | | | | | | |
| NaCl, 0.6 mg/ml | | | | | | | | |
| polysorbate 80 | | | | | | | | |

T0-indicates a value at zero time point

### Example 2: Buffer Free High Concentration Vedolizumab Formulations with Amino Acids

As part of the experimental design, to prepare a high concentration water based vedolizumab formulation, purified high concentration vedolizumab antibody at a concentration of approximately 100 mg/ml in arginine histidine buffer back ground was obtained from downstream chromatographic steps. Post which, depending on the requirement of excipients in the final formulation, the vedolizumab antibody was buffer exchanged with a composition comprising water, arginine and NaCl, until vedolizumab antibody in histidine buffer was completely exchanged with water. Post buffer exchange, the formulation was spiked with PEG-400 and the sample was concentrated upto 175 mg/ml. Post which, polysorbate 80 was spiked in the formulations.

To maintain control, approximately 100 mg/ml of purified vedolizumab in histidine buffer back ground containing 26.3 mg/ml arginine, 100 mg/ml sucrose was obtained from downstream chromatographic steps was buffer exchanged with a composition containing histidine buffer, arginine, and citrate. Post which, the antibody was concentrated upto 175 mg/ml. Polysorbate-80 was added to the final formulation. Approved high concentration liquid vedolizumab formulation contains the above composition. Hence, maintained as control.

Details of the two vedolizumab formulations are mentioned in Table 2. All vedolizumab formulations were subjected for accelerated stability studies at 40° C. for four weeks and at 25° C. for four weeks. Post which, the samples were analyzed for high molecular weight (HMW) species and monomer content using size exclusion chromatography (SEC) [results are given in Table 3 and Table 4] and also checked for main peak content, and acidic variants using ion-exchange chromatography [Table 5 and Table 6].

TABLE 2
Compositions of various high concentration vedolizumab
formulations prepared as per example 2

| Sample | |
|---|---|
| Name | Composition |
| Vmab-C | Vedolizumab 175 mg/ml, 50 mM histidine |
| | monohydrochloride, arginine•HCl 26.3 mg/ml, 6.7 mg/ml |
| | sodium citrate, 0.5 mg/ml citric acid monohydro chloride, |
| | 0.6 mg/mL polysorbate 80, pH 6.2 |
| Vmab-1 | Vedolizumab 175 mg/ml, 50 mM NaCl, water, 26 mg/ml |
| | arginine•HCl, 10% PEG-400, 0.6 mg/mL polysorbate 80, |
| | pH 6.2 |

TABLE 3
SEC data of high concentration vedolizumab formulations prepared as per
example 2 at 40° C. for four weeks

| | SEC data at 40° C. | | | | | | | | | | | |
|---|---|---|---|---|---|---|---|---|---|---|---|---|
| Sample | % of LMW at 40° C. | | | | % of monomer at 40° C. | | | | % of HMW | | | |
| Name | 0 W | 1 W | 2 W | 4 W | 0 W | 1 W | 2 W | 4 W | 0 W | 1 W | 2 W | 4 W |
| Vmab- | 0.07 | 0.4 | 0.4 | 0.9 | 99.47 | 99.0 | 98.7 | 97.9 | 0.5 | 0.7 | 0.9 | 1.2 |
| Control | | | | | | | | | | | | |
| Vmab-1 | 0.11 | 0.4 | 0.5 | 0.7 | 99.22 | 98.6 | 98.1 | 97.3 | 0.7 | 1.1 | 1.4 | 2.0 |

W-indicates weeks,

TABLE 4
SEC data of high concentration vedolizumab formulations prepared as per
example 2 at 25° C. for four weeks

| | SEC data at 25° C. | | | | | | | | |
|---|---|---|---|---|---|---|---|---|---|
| Sample | % of LMW at 25° C. | | | % of monomer at 25° C. | | | % of HMW at 25° C. | | |
| Name | 0 W | 4 W | Δ 4 W | 0 W | 4 W | Δ 4 W | 0 W | 4 W | Δ 4 W |
| Vmab- | 0.1 | 0.2 | 0.1 | 99.5 | 99.1 | 0.4 | 0.5 | 0.7 | 0.2 |
| Control | | | | | | | | | |
| Vmab-1 | 0.1 | 0.3 | 0.2 | 99.2 | 98.5 | 0.7 | 0.7 | 1.2 | 0.5 |

W-indicates weeks, Δ-indicates change

TABLE 5
IEX data of high concentration vedolizumab formulations
prepared as per example 2 kept at 40° C. for four weeks

| | IEX data at 40° C. | | | | | | | |
|---|---|---|---|---|---|---|---|---|
| | % of Acidic variants | | | | % of main peak | | | |
| Sample | at 40° C. | | | | at 40° C. | | | |
| Name | 0 W | 1 W | 2 W | 4 W | 0 W | 1 W | 2 W | 4 W |
| Vmab-C | 21.9 | 25.6 | 31.4 | 45.3 | 68.1 | 57.3 | 52.1 | 42.5 |
| Vmab-1 | 23.1 | 22.3 | 24.5 | 33.0 | 67.3 | 54.6 | 50.1 | 54.6 |

W-indicates weeks

TABLE 6
IEX data of high concentration vedolizumab formulations prepared
as per example 2 kept at 25° C. for four weeks

| | IEX data at 25° C. | | | | | |
|---|---|---|---|---|---|---|
| | % of Acidic variants | | | % of main peak | | |
| Sample | at 25° C. | | | at 25° C. | | |
| Name | 0 W | 4 W | Δ 4 W | 0 W | 4 W | Δ 4 W |
| Vmab-C | 21.9 | 24.4 | 2.5 | 68.1 | 64.9 | −3.2 |
| Vmab-1 | 23.1 | 23.7 | 0.6 | 67.3 | 63.3 | −4.0 |

W-indicates weeks, Δ-indicates change

All the above formulations were also checked for change in pH. It was observed that there is no change in pH of the formulations even after storage for four weeks at 40° C. and also at 25° C.

Further, all the samples were checked for visible particles. It was observed that, all the samples were clear, colorless without any visible particles.

### Example 3: Buffer Free High Concentration Vedolizumab Formulations in Water

To prepare a buffer free 160 mg/ml vedolizumab formulation, approximately 60-70 mg/ml vedolizumab in a buffer composition comprising histidine-phosphate buffer, trehalose, arginine and surfactant obtained from downstream chromatographic steps. The obtained vedolizumab sample was buffer exchanged at least three times with a buffer free composition comprising water. Post which, the diafiltered vedolizumab in water was subjected for ultrafiltration to concentrate upto 175 mg/ml. Post which, polysorbate-80 was added to the highly concentrated vedolizumab in water. Buffer based vedolizumab formulation at a concentration of ˜160 mg/ml in a buffer composition comprising histidine buffer, arginine, citrate and polysorbate was used as control. The approved liquid vedolizumab formulation contains the same composition. Hence, buffer based vedolizumab formulation contains the same.

Post which, buffer based and buffer free vedolizumab formulations were subjected for accelerated stability studies at 25° C. for one week and formulations were analyzed for high molecular weight content, monomer using size exclusion chromatography (SEC) and also checked for acidic variants and main peak content of using ion-exchange ion chromatography. Results are given in below Table 7 and 8.

TABLE 7
Formulation composition and SEC data of vedolizumab
formulations prepared as per example-3 when stored at
25° C. for one week.

| | | | High | | Low | |
|---|---|---|---|---|---|---|
| | | | molecular | | molecular | |
| | Monomer | | weight | | weight | |
| Sample ID and | content | | species | | species | |
| Formulation | at 25° C. | | at 25° C. | | at 25° C. | |
| Composition | T0 | T1 W | T0 | T1 W | T0 | T1 W |
| Vmab-C 156.9 mg/ml | 99.4 | 99.3 | 0.6 | 0.6 | 0.1 | 0.1 |
| vedolzumab, 5.5 mM | | | | | | |
| Citric acid monohydrate, | | | | | | |
| 20.6 mM Tri Sodium | | | | | | |
| citrate dehydrate, 38.8 | | | | | | |
| mM L-histidine, 9.2 mM | | | | | | |
| L-histidine | | | | | | |
| monohydrochloride, | | | | | | |
| 128.6 mM L-arginine | | | | | | |
| hydrochloride, 2 mg/mL | | | | | | |
| polysorbate 80 | | | | | | |
| Vmab 2-173.9 mg/ml | 99.0 | 98.9 | 0.9 | 1.0 | 0.1 | 0.1 |
| vedolzumab, 2 mg/ml | | | | | | |
| polysorbate 80 and water | | | | | | |

TABLE 8
IEX data of vedolizumab formulations prepared as
per example-3 when stored at 25° C. for one week.

| | % Acidic variants at 25° C. | | % main peak at 25° C. | |
|---|---|---|---|---|
| Sample ID | T0 | T1 W | T0 | T1 W |
| Vmab-C | 72.3 | 70.9 | 17.7 | 18.7 |
| Vmab 2 | 71.0 | 64.8 | 16.3 | 16.3 |

### Example 4: Buffer Free Formulation of Vedolizumab at about 60 mg/ml Concentration

To prepare a buffer free 60 mg/ml vedolizumab formulation purified vedolizumab antibody at a concentration of approximately 60 mg/ml to 70 mg/ml in a buffer composition comprising histidine-phosphate buffer, arginine and sugar was obtained from downstream chromatographic steps. Post which, the vedolizumab antibody was buffer exchanged with a buffer free composition comprising water until vedolizumab antibody in histidine-phosphate buffer was completely exchanged with water until vedolizumab was completely transferred in water and concentration of the antibody was between 55 to 70 mg/ml. Post which, polysorbate-80 was added to the buffer exchanged vedolizumab in water sample. Buffer based vedolizumab formulation at a concentration of ˜60 mg/ml in histidine-phosphate buffer background comprising arginine, trehalose and polysorbate was used as control [formulation composition given in Table 9].

Post which, buffer based and buffer free vedolizumab formulations were subjected for accelerated stability studies at 25° C. for one week and formulations were analyzed for high molecular weight (HMW) content, monomer, low molecular weight (LMW) content using size exclusion chromatography (SEC) and also checked for change in pH. Results are given in below Table 9.

TABLE 9
Formulation composition and quality attributes of vedolizumab
formulations prepared as per example-4 when stored at 25° C.
for one week.

| | | | High | | | | | |
|---|---|---|---|---|---|---|---|---|
| | | | molecular | | Low | | | |
| | Monomer | | weight | | molecular | | | |
| | content | | species | | weight | | pH at | |
| Formulation | at 25° C. | | at 25° C. | | at 25° C. | | 25° C. | |
| Composition | T0 | T1 W | T0 | T1 W | T0 | T1 W | T0 | T1 W |
| 58 mg/ml | 99.4 | 99.4 | 0.5 | 0.5 | 0.1 | 0.1 | 6.2 | 6.1 |
| vedolzumab, 20 | | | | | | | | |
| mM Histidine | | | | | | | | |
| phosphate, 12 | | | | | | | | |
| mg/ml Arginine | | | | | | | | |
| HCl, 75 mg/ml | | | | | | | | |
| trehalose, 2.92 | | | | | | | | |
| mg/ml NaCl and | | | | | | | | |
| 0.6 mg/ml | | | | | | | | |
| polysorbate-80 | | | | | | | | |
| 69 mg/ml | 99.2 | 97.0 | 0.8 | 0.7 | 0.1 | 2.3 | 6.1 | 5.9 |
| vedolzumab, 2 | | | | | | | | |
| mg/ml polysorbate | | | | | | | | |
| 80 and water | | | | | | | | |

1. An aqueous buffer free high concentration α4β7 antibody formulation, comprising an α4β7 antibody, water and surfactant, stabilized at a pH of 6.0-6.5.

2. The formulation as claimed in claim 1, has the antibody concentration from about 50 mg/ml to about 200 mg/ml.

3. (canceled)

4. (canceled)

5. The formulation as claimed in claim 1, which further comprises one or more pharmaceutically acceptable excipients, wherein the excipients include polyethylene glycol, salt, or arginine.

6. The formulation as claimed in claim 1, which exhibits stability by maintaining >95% or more of the antibody in monomeric form, when the formulation is stored at 40° C. for one to four weeks or at 25° C. for one to four weeks.

7. A method of stabilizing α4β7 antibody in an aqueous solution, comprising;
a) expressing and purifying an α4β7 antibody,
b) subjecting the purified antibody to diafiltration with a buffer free diafiltration medium comprising water, to obtain the α4β7 antibody in solution,
c) concentrating the diafiltered antibody up to 200 mg/ml by ultrafiltration,
d) followed by formulating the antibody in water, to obtain a highly concentrated α4β7 antibody solution, wherein the antibody is stable at room temperature.

8. The method as claimed in claim 7, further comprise polyethylene glycol and salt added to diafiltration medium of step b).

9. A method of reducing formation of acidic variants of an α4β7 antibody in an aqueous formulation composition of the antibody, by wherein the method comprises, formulating the antibody in a composition comprising water, and surfactant, and wherein the composition is maintained to a pH value of 6.0 to 6.5 without any buffering agent.

10. The formulation as claimed in claim 1, wherein the α4β7 antibody is vedolizumab.

11. The formulation as claimed in claim 5, which exhibits stability by maintaining >95% or more of the antibody in monomeric form, when the formulation is stored at 40° C. for one to four weeks or at 25° C. for one to four weeks.

12. The method as claimed in claim 7 wherein the α4β7 antibody is vedolizumab.

13. The method as claimed in claim 9 wherein the α4β7 antibody is vedolizumab.
